(19) United States
(12) Patent Application Publication
(10) Pub. No.: US 2010/0174586 A1
(43) Pub. Date: Jul. 8, 2010

(54) Methods for Measuring Emotive Response and Selection Preference

(76) Inventors: Charles John Berg, JR., Wyoming, OH (US); David Keith Ewart, Uxbridge (GB); Nick Robert Harrington, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/726,658

(22) Filed: Mar. 18, 2010

Related U.S. Application Data
(63) Continuation of application No. 11/851,638, filed on Sep. 7, 2007.

(51) Int. Cl. G06Q 10/00; G06Q 50/00; A61B 5/00; A61B 5/11

(52) U.S. Cl. 705/10; 600/301; 600/595

(57) ABSTRACT

The present invention relates generally to consumer research methods for measuring emotive response to visual stimuli.

### CROSS REFERENCE TO RELATED APPLICATION

This application claims the benefit of U.S. Provisional Application No. 60/842,757, filed Sep. 7, 2006; U.S. Provisional Application No. 60/842,755, filed Sep. 7, 2006; U.S. Provisional Application No. 60/885,998, filed Jan. 22, 2007; U.S. Provisional Application No. 60/886,004, filed Jan. 22, 2007 and U.S. Utility application Ser. No. 11/851,638, filed Sep. 7, 2007.

### FIELD OF THE INVENTION

The present invention relates generally to methods for measuring emotive response and selection preference in situations involving at least one visual stimulus and product usage or selection. In particular, where at least one visual stimulus is involved, the present invention relates to methods of using an emotive response and selection preference system comprising at least one eye-tracking or head-tracking apparatus, at least one physiological apparatus, and at least one visual stimulus, to obtain consumer feedback regarding their selection preference or determine their probable emotive state in response to the at least one visual stimulus.

### BACKGROUND OF THE INVENTION

The commercial success of a consumer product is dependent, at least in part, upon the manner in which it evokes a positive response from a consumer. As a result, millions of dollars are spent on consumer research by companies. One tool used is consumer analysis of which there are multiple consumer analysis models to choose from. For example, two areas of frequent study are shopper analysis and user analysis.

Under a shopper analysis model, one example involves the interaction of three elements by a consumer: (1) an attention element used to gather information about a consumer product, e.g., by physically or virtually observing the packaging or display of a product on a retail shelf; (2) an opinion formation element involving an emotive response to the consumer product; and, (3) a probable choice decision element on whether to use, not use, recommend, not recommend, select or not select for purchase. The data, however, obtained from this consumer analysis model has been inefficient and inaccurate. One shortcoming is that frequently the mental and emotional processing by the consumer, in response to the visual stimulus of a product, package, display, etc., occurs at a sub-conscious level rather than through a deliberate conscious process. Some of the processing and response to a visual stimulus manifests itself into emotive states or feelings which the person then detects, or feels.

For example, a store or partial store simulation (physical, virtual or a combination) can be used to evaluate a consumer reaction to a consumer product on a retail store shelf. The consumer observes or interacts with the product and then gives feedback. The consumer can provide written or oral feedback in response to a live questioner or pre-recorded questions (written or oral). Such feedback may include the appeal of the product, how they feel about the offering and whether they might purchase or use the product, among others.

While this technique provides some value, it can also provide misleading and inaccurate consumer market prediction data. One reason for the inaccuracy is that a consumer's analysis of a product is faster and more covert than can be accurately measured today. For example, consumers typically rely on sub-conscious, as well as conscious, thought processes. Current techniques used in the shopper analysis model tend to evaluate the selection preference of the consumer by heavily focusing on the conscious processes, e.g., asking the consumer various questions relating to the product which requires the consumer to consciously provide an answer. Obtaining direct feedback introduces a level of consciousness that is typically not present in the actual shopping experience. For instance, the use of conscious inquiry requires the consumer to accurately recall and capture, to the best of their ability, their emotive state in precise terms seconds, minutes or hours after interacting with the consumer product.

Another common technique used in consumer research is the user analysis model. It involves the interaction of four steps by a consumer: (1) at least one real or prototypical product is given to or selected by a consumer; (2) optional instructions are given or selected by the consumer; (3) the consumer uses the products; and, (4) the consumer provides feedback about likes, dislikes, and observations of the consumer either during use, just after use or later, can be obtained. The data, however, obtained from the user analysis model has also been inefficient, incomplete and/or inaccurate. Similar to the shortcomings of the shopper analysis model, much of the mental and emotional processing of the consumer occurs at a sub-conscious level rather than through a deliberate conscious process, e.g., the consumer experiences various emotive states. Moreover, the user analysis model evaluates the selection preference of the consumer by heavily focusing on conscious processes, e.g., presenting the same drawbacks associated with the shopper analysis model.

For some or many consumer analyses techniques, understanding eye-gaze position can be beneficial. Under a consumer analysis model that focuses primarily on visual stimuli, for instance under a shopper analysis model, eye-tracking techniques are employed to gather data about the attention element. Typically, shoppers wear an eye-tracking apparatus on their head and a computer system combines the detected eye-gaze position data rates to the available viewing area, as also gathered via video camera affixed to the eye-tracking apparatus. This technique allows a researcher to view and record when and where, e.g., to which visual point, a consumer's eye-gaze is directed to, and how long they spend at each point in the available viewing area. Fix-mounted remote eye-gaze sensors have also been used with a virtual stimulus such as a flat-screen monitor that displays a consumer product. The attention element of the shopper analysis model is only one element. Another critical element is the consumer's emotive response.

The emotive response element is much more difficult to assess since conscious and sub-conscious decisions guide a consumer's reaction to a product, such as in visual stimulus situations, use experience situations, and in product beneficiary situations. It is well-known that certain emotions can invoke one or more physiological responses. For example, when a person is in a fearful state, their heart rate tends to increase and their muscles may involuntary contract. Another example is when a person is in a calm state, their respiratory functions can retard, including their heart rate, and their muscles may involuntarily become more flexible and loose. A consumer may not provide or accurately articulate this type of emotive feedback, e.g., their emotive state, in the consumer analyses models since they may not even consciously be aware of the invoked emotive state.

For example, the smell of a product may sub-consciously invoke nostalgia within the consumer, and, the consumer may use that product solely based on feelings of nostalgia that they cannot consciously articulate. Similarly, the smell of a product may invoke a sub-conscious emotive state of fear, and the consumer may not like the product, and again cannot consciously articulate the reasons behind their selection preference. As a result, current techniques may not provide sufficient accuracy in measuring selection preference of a product by a consumer.

Accordingly, there is a need for systems and methods for measuring emotive response and selection preference that can provide accurate consumer feedback, whether conscious or sub-conscious, relating to a company's products for purposes of conducting consumer research, such as for shopping, usage analysis, and product beneficiary analysis. There is also a need for providing improved and more accurate consumer analyses models that avoid inaccuracies and inefficiencies associated with current methods.

See e.g., US 2003/0032890; US2003/0236451; US 2005/0243054; US 2005/0289582; U.S. Pat. No. 5,676,138; U.S. Pat. No. 6,190,314; U.S. Pat. No. 6,309,342; U.S. Pat. No. 6,572,562; U.S. Pat. No. 6,638,217; U.S. Pat. No. 7,046,924; U.S. Pat. No. 7,249,603; WO 97/01984; WO 2007/043954; and Lindsey, Jeff; www.jefflindsay.com/market-research.shtml entitled “The Historic Use of Computerized Tools for Marketing and Market Research: A Brief Survey.”

### SUMMARY OF THE INVENTION

In general, the present invention, and in various exemplary embodiments, provides a method of using an emotive response and selection preference system. In one embodiment, a method of conducting consumer research is provided comprising: providing at least one visual stimulus to a consumer; measuring the consumer's response to the at least one visual stimulus with an eye-tracking apparatus; measuring the consumer's response to the at least one visual stimulus with a physiological apparatus; converting the measured physiological data to a probable emotive state of the consumer; and, synchronizing said converted physiological data and the measured eye-tracking data.

In another embodiment, a method of identifying the probable emotive state of a consumer is provided comprising: providing at least one visual stimulus to the consumer; eye-tracking at least one eye movement of the consumer in response to the provided visual stimulus; physiologically measuring at least one physiological change of the consumer in response to the provided visual stimulus; and synchronizing the eye-tracking data and physiologically measured data to identify the probable emotive state of the consumer.

In still yet another embodiment, a method of conducting consumer research is provided comprising: providing at least one visual stimulus to the consumer; obtaining at least a first data set by measuring the consumer's response to the at least one visual stimulus with an eye-tracking apparatus; obtaining at least a second data set by measuring the consumer's response to the at least one visual stimulus with a physiological apparatus; and synchronizing said first and second data steps.

Another embodiment provides for a method of obtaining consumer research data comprising the steps: presenting a visual stimulus to a consumer; collecting eye gazing data in a non-tethered manner from the consumer while presenting the visual stimulus to the consumer; collecting non-ocular biometric data in a non-tethered manner from the consumer while presenting the visual stimulus to the consumer.

Another embodiment provides for a method of obtaining consumer research data comprising the steps: presenting a visual stimulus to a consumer; defining an area of interest (AOI) in the visual stimulus; collecting eye gazing data from the consumer while presenting the visual stimulus to the consumer and with regard to the AOI; collecting biometric data from the consumer while presenting the visual stimulus to the consumer; and associating the collected biometric data and the collected eye gazing data regarding the AOI.

Another embodiment provides for a method of obtaining consumer research data comprising the steps: presenting a visual stimulus to a consumer; defining an area of interest (AOI) in the visual stimulus; collecting eye gazing data from the consumer while presenting the visual stimulus to the consumer and with regard to the AOI; collecting biometric data from the consumer while presenting the visual stimulus to the consumer; and translating the collected biometric data to an emotional metric data; and associating the emotional metric data and the collected eye gazing data regarding the AOI.

Another embodiment provides for a report comprising: a graphic of a visual stimulus; an area of interest (AOI) indicium on the graphic; an emotional metric data indicium or a biometric data indicium on the graphic and in relation to the AOI indicium; an eye gazing indicium on the graphic and in relation to the AOI.

Another embodiment provides for a method of obtaining consumer research data comprising the steps: presenting a visual stimulus to a consumer; collecting face direction data in a non-tethered manner from the consumer while presenting the visual stimulus to the consumer; collecting non-ocular biometric data in a non-tethered manner from the consumer while presenting the visual stimulus to the consumer.

Systems and software are also provided.

### BRIEF DESCRIPTION OF THE DRAWINGS

The above and other advantages and features of the invention will be more clearly understood from the following detailed description which is provided in connection with the accompanying drawings.

FIGS. 1A-1E are diagrams of various embodiments of the emotive response and selection preference system that may be used with the methods of the present invention.

### DETAILED DESCRIPTION OF THE INVENTION

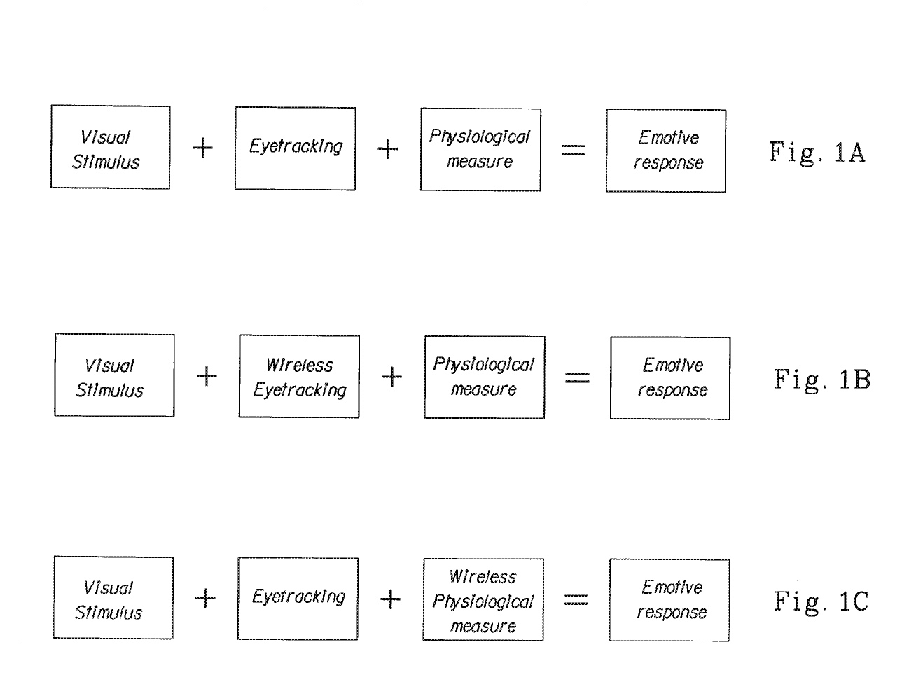

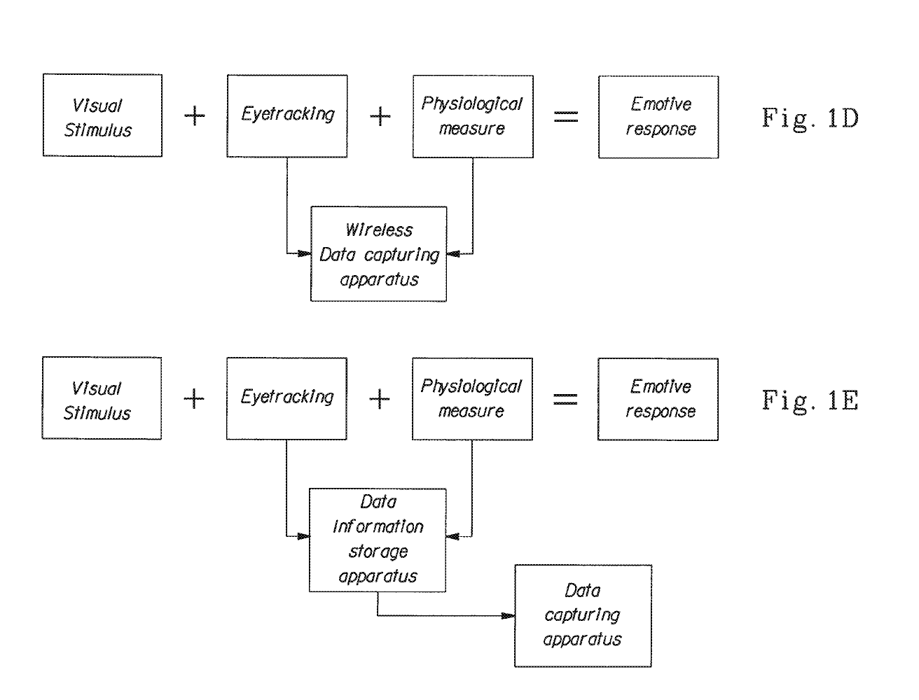

In the following detailed description, reference is made to the accompanying drawings (FIGS. 1A-1E) which form a part hereof and illustrate specific exemplary embodiments by which the invention may be practiced. It should be understood that like reference numerals represent like elements throughout the drawings (FIGS. 1A-1E). These embodiments are described in sufficient detail to enable those skilled in the art to practice the invention. It is to be understood that other embodiments may be utilized, and that structural, logical, chemical, biological and electrical changes, including the omission, addition, or departure from the sequence of steps disclosed in a method, may be made without departing from the spirit and scope of the present invention.

At the outset, it should be appreciated that although the methods of the present invention are described as being used for consumer research in a retail environment, the methods can be used to conduct research in any environment. For example, consumer research can be conducted in a consumer's home, while a consumer watches television, while a consumer goes throughout their normal daily activities, including but not limited to waking up, cleansing, brushing their teeth, combing their hair, washing their hair, cleaning their clothes, driving, going to work, eating lunch, and the like. In short, the methods of the present invention are applicable to any situation where consumer research is desired.

All ranges given herein include the end of the ranges and also all the intermediate range points.

The term “comprising” refers to various components or steps that may be conjointly employed, although additional steps or components may be utilized, if desired. Accordingly, the term “comprising” may encompass the more restrictive terms “consisting essentially of” and “consisting of”.

The term “consumer(s)” is used in the broadest sense and is a mammal, usually human, that includes but is not limited to a shopper, user, beneficiary, or an observer or viewer of products or services by at least one physiological sense such as visually by magazines, a sign, virtual, TV, or, auditory by music, speech, white noise, or olfactory by smell, scent, insult; or, by tactile, among others. A consumer can also be involved in a test (real world or simulation) whereas they may also be called a test panelist or panelist. In one embodiment, the consumer is an observer of another person who is using the product or service. The observation may be by way of viewing in-person or via photograph or video.

The term “shopper” is used in the broadest sense and refers to an individual who is considering the selection or purchase of a product for immediate or future use by themselves or someone else. A shopper may engage in comparisons between consumer products. A shopper can receive information and impressions by various methods. Visual methods may include but are not limited to the product or its package within a retail store, a picture or description of a product or package, or the described or imaged usage or benefits of a product on a website; electronic or electrical media such as television, videos, illuminated panels & billboards & displays; or, printed forms such as ads or information on billboards, posters, displays, “Point-of-purchase” POP materials, coupons, flyers, signage, banners, magazine or newspaper pages or inserts, circulars, mailers, etc. A shopper sometimes is introduced into a shopping mode without prior planning or decision to do so such as with television program commercials, product placement within feature films, etc. For brevity, the shopper/consumer/panelist may be referred to as “she” for efficiency but will collectively include both female and male shoppers/consumers/and panelists.

The term “viewer” is used in the broadest sense and refers to a recipient of visual media communication where the product is entertainment information including information needed for decisions or news. Similar to the shopper examples, visual methods may include but are not limited to websites; electronic or electrical media such as television, videos, illuminated panels & billboards & displays; or, printed forms. The visual media can be supplemented with other sensorial stimulus such as auditory, among others.

The term “consumer analysis” is used in the broadest sense and refers to research involving the consumer reacting to in relation to a company's products such as in shopping, usage, post-application benefits receipt situations. Many current techniques, with significant drawbacks, exist to attempt to understand the emotive response or selection interest in one or more products, or a task involving one or more products. See e.g., US 2007/0005425.

The term “product(s)” is used in the broadest sense and refers to any product, product group, services, communications, entertainment, environments, organizations, systems, tools, and the like. For example, an example of a product group is personal and household products, such as used by a person, family or household. Examples of a representative, and non-limiting list of product categories within the personal and household product group includes antiperspirants, baby care, colognes, commercial products (including wholesale, industrial, and commercial market analogs to consumer-oriented consumer products), cosmetics, deodorants, dish care, feminine protection, hair care, hair color, health care, household cleaners, laundry, oral care, paper products, personal cleansing, disposable absorbent articles, pet health and nutrition, prescription drugs, prestige fragrances, skin care, foods, snacks and beverages, special fabric care, shaving and other hair growth management products, small appliances, devices and batteries, services such as haircutting, beauty treatment, spa treatment, medical, dental, vision services, entertainment venues such as theaters, stadiums, as well as entertainment services such as film or movie shows, plays and sporting events A variety of product forms may fall within each of these product categories.

Exemplary product forms and brands are described on The Procter & Gamble Company's website www.pg.com, and the linked sites found thereon. It is to be understood that consumer products that are part of product categories other than those listed above are also contemplated by the present invention, and that alternative product forms and brands other than those disclosed on the above-identified website are also encompassed by the present invention.

Exemplary products within the laundry category include detergents (including powder, liquid, tablet, and other forms), bleach, conditioners, softeners, anti-static products, and refreshers (including liquid refreshers and dryer sheets). Exemplary products within the oral care category include dentifrice, floss, toothbrushes (including manual and powered forms), mouth rinses, gum care products, tooth whitening products, and other tooth care products. Exemplary feminine protection products include pads, tampons, interlabial products, and pantiliners. Exemplary baby care products include diapers, wipes, baby bibs, baby change and bed mats, and foaming bathroom hand soap.

Exemplary health care products include laxatives, fiber supplements, oral and topical analgesics, gastro-intestinal treatment products, respiratory and cough/cold products, heat delivery products, and water purification products. Exemplary paper products include toilet tissues, paper towels, and facial tissues. Exemplary hair care products include shampoos, conditioners (including rinse-off and leave-in forms), and styling aids. Exemplary household care products include sweeper products, floor cleaning products, wood floor cleaners, antibacterial floor cleaners, fabric and air refreshers, and vehicle washing products. Skin care products include, but are not limited to, body washes, facial cleansers, hand lotions, moisturizers, conditioners, astringents, exfoliation products, micro-dermabrasion and peel products, skin rejuvenation products, anti-aging products, masks, UV protection products, and skin care puffs, wipes, discs, clothes, sheets, implements and devices (with or without skin care compositions).

Other product groups include but are not limited to: sports equipment, entertainment (books, movies, music, etc), vision, and in-home-consumed medical and first aid, among others.

The term “emotive response indicator(s)” refers to a measure of a physiological or biological process or state of a human or mammal which is believed to be linked or influenced at least in part by the emotive state of the human or mammal at a point or over a period of time. It can also be linked or influenced to just one of the internal feelings at a point or period in time even if multiple internal feelings are present; or, it can be linked to any combination of present feelings. Additionally, the amount of impact or weighting that a given feeling influences an emotive response indicator can vary from person-to-person or other situational factors, e.g., the person is experiencing hunger, to even environmental factors such as room temperature.

The term “emotive state(s)” refers to the collection of internal feelings of the consumer at a point or over a period of time. It should be appreciated that multiple feelings can be present such as anxiousness and fear, or anxiousness and delight, among others.

The term “imaging apparatus” is used in the broadest sense and refers to an apparatus for viewing of visual stimulus images including, but not limited to: drawings, animations, computer renderings, photographs, and text, among others. The images can be representations of real physical objects, or virtual images, or artistic graphics or text, and the like. The viewable images can be static, or dynamically changing or transforming such as in sequencing through a deck of static images, showing motions, and the like. The images can be presented or displayed in many different forms including, but not limited to print or painted media such as on paper, posters, displays, walls, floors, canvases, and the like. The images can be presented or displayed via light imaging techniques and displayed for viewing by the consumer on a computer monitor, plasma screen, LCD screen, CRT, projection screen, fogscreen, water screen, VR goggles, headworn helmets or eyeglasses with image display screens, or any other structure that allows an image to be displayed, among others. Projected imagery “in air” such as holographic and other techniques are also suitable.

The consumer-viewed scale of the imagery and its elements can be of any size or proportion. For example, outside of an in-store environment, the imaging apparatus can comprise a high-resolution and large-scale imaging apparatus to display virtual images of prospective or current product shelf arrangements (so called product arrays) for the purpose to use the virtual stimulus to conduct consumer research regarding consumer products sold in a retail environment. The virtual imaging apparatus can comprise a semi-transparent screen, banner, or a specially designed consumer product alone or in combination, that can reflect or project an image, with additional elements such as sound, aesthetic enhancements, aromatherapy or fragrances, and a feedback apparatus that draws or enhances a consumer's attention to a company's particular product in an in-store environment. The virtual imaging apparatus can also comprise a computer monitor, plasma screen, LCD screen, CRT, image projection unit, fogscreen, water screen, holographic or any other structure that allows a real or virtual image to be displayed. It can also comprise immersive or partially immersive systems such as large and physical, multi-screen chambers like the CAVE which are currently sold by Fakespace Systems Inc., or simulators such as used for simulation of transportation experiences, or VR (virtual reality) goggles, helmets and masks.

For example, a high-resolution, large-scale imaging apparatus (imaging apparatus) can be used by a company to create an in vitro virtual environment simulating an in-store retail environment to evaluate merchandising scenarios and/or consumer packaging graphics proposals. The imaging apparatus allows a company to simulate a shelf or plurality of shelves of an in-store retail store on about a 1:1 scale. For instance, the imaging apparatus can display a plurality of consumer products, e.g., a company's and competitor's products. The imaging apparatus can be a single imaging apparatus or a plurality of imaging apparatuses which can be used to create an in-store virtual environment. There are many variations and uses of the imaging apparatus that are contemplated. In one embodiment, the image comprises a high resolution, wherein the high resolution is greater than about 1 megapixels, alternatively greater than 2 megapixels, 5 megapixels, or 10 megapixels, or 15 megapixels, or 20 megapixels, or greater than 25 megapixels. Without wishing to be bound by theory, the greater resolution allows for more “life like” experience for the consumer with the speed and efficiency that a virtual image provides. For comparison, current “HDTV” is generally about 1-2 megapixels. In another embodiment the object(s) of the virtual image is in a ratio at or greater than 1:1, alternatively greater than 2:1, to that of the same object as a non-virtual image, respectively.

The imaging apparatus can also include physical cues to enhance the immersive feel such as a physical aisle navigation sign overhead, or a soundtrack of typical in-store sounds. The imaging apparatus allows a company to generate virtual images of various prototypes without physically having to make them, or, to provide various color schemes, advertising slogans, and/or arrangement of the products on a shelf. The imaging apparatus can provide stationary images, or, images that scroll simulating a consumer walking in an in-store environment. Alternatively, a moving floor is provided with a stationary imaging apparatus allowing a consumer to physically move, e.g., walk, while images on the imaging apparatus change or stay stationary as the consumer moves. In addition, the imaging apparatus can be a touch-screen imaging apparatus allowing a consumer to physically touch the screen and thereby rotate, magnify, and manipulate, among other things, a consumer product, as if they were in a retail store, if desired.

The screen can display images that are 3D, 2D, or a combination of both. The large screen imagery can be controlled by pre-program, observers or optionally controlled by the consumer, i.e., self-navigation, through their dynamic movement or interaction (touch-screen, wand, joystick, or any other element). In essence, the consumer is given a control object that allows them to manipulate the images on the screen in real-time.

The imaging apparatus can also be configured or combined with other apparatuses to sense the body position of the consumer using sensing aids, or, without sensing aids. The sensing aids can be worn or mounted on the consumer's body or remotely-located. Alternatively, the images on the screen can move as the consumer walks on a sliding surface, e.g., conveyer belt or moving walkway, and the sliding surface optionally can control the movement of images on the screen. For example, as the consumer stops, the projected images stop, and, when the consumer continues to walk, the images continuously and preferably seamlessly change simulating as if the consumer is walking down an aisle in a retail store. See e.g., US 2007/0069021. Other techniques to allow the consumer to physically move their legs, but maintain a relatively stationary position to the imaging apparatus may include: moving motorized robot tiles system consisting of small floor squares controlled by robots anticipating a where a consumer will step and then move backwards as the consumer steps forward thereby keeping the consumer stationary as the consumer walks; or an advanced “roller skate” comprising shoes with small long rollers (e.g., cylindrical bearings) that are motorized to resist a consumer's forward motion to mimic the resistance associated with walking

The imaging apparatus also allows, if desired, a consumer to examine a product closely. A wand, an electronic device, or the screen itself can be responsive to a consumer's touch. In other words, the consumer can carry out similar movements they would do as if they were physically in a store. The consumer can pick up a 3D representation of the product, rotate it, and put it in a virtual cart, or real-life cart, if desired. The cart can also be a haptic cart where, for example through the handlebars, it provides a force or force feedback to better simulate the pushing or pulling of a cart along a floor. The consumer can pull out a single product, or, pull out a plurality of products and hold them out for view. A sensor is used that sends the translation and rotation values to a separate electronic system, that calculates real-time object movements conducted by the consumer. An example of a means for displaying a virtual reality environment, as well as receiving feed-back response to the environment, is described in U.S. Pat. No. 6,425,764; and US 2006/0066509 A1. The imaging apparatus can be part of a system whereas 3D space synchronization to correspond eye-gaze direction with the visual stimulus can be executed by: (i) tracking head position (or face position) in 3D space either separately or as a derivation of eye-gaze tracking; (ii) tracking eye gaze direction; and (iii) having digitally mapped 3D space (or space-time) coordinates for the elements in the display.

In one embodiment, a method is provided the steps: presenting a visual stimulus to a consumer; collecting head position tracking and/or face direction tracking of the consumer while presenting the visual stimulus to the consumer; optionally collecting eye gazing data from the consumer while presenting the visual stimulus to the consumer; collecting biometric data from the consumer while presenting the visual stimulus to the consumer. For purposes of the present invention, the term “face direction data” means determining the field of view the consumer's face is facing from the wholly available visual environment surrounding the consumer. Without wishing to be bound by theory, this approach provides an estimation (for the sake of efficiency) of whether the consumer is viewing the visual stimulus (including any AOI's). Face direction data can be gathered by various known means including head position tracking, and face tracking. For example, face direction data may be obtained by remote video tracking means, by remote electromagnetic wave tracking, or by placing fixed sensor(s) or tracking point(s) at or near the consumer's head or face.

The term “visual stimulus” is used in the broadest sense and refers to any virtual or non-virtual image including but not limited to a product, object, stimulus, and the like, that an individual may view with their eyes. In one embodiment, a non-visual stimulus (e.g., smell, sound, and the like) is substituted for the visual stimulus or is presented concurrently/concomitantly with the visual stimulus. Examples of smells or aromas are described in WO 2007/075205 (pg. 8); U.S. Pat. No. 6,280,751; US 2004/0071757. In one embodiment, the visual stimulus may be archived as a physical image (e.g., photograph) or digital image for analysis or even presentation (such as a report).

The term “physiological measurement(s)”, as used herein, broadly includes both biological measures as well as body language measures which measure both the autonomic responses of the consumer, as well as learned responses whether executed consciously or sub-consciously, often executed as a learned habit. Physiological measurements are sometimes referred to as “biometric expressions” or “biometric data.” See e.g., U.S. Pat. No. 5,676,138; U.S. Pat. No. 6,190,314; U.S. Pat. No. 6,309,342; U.S. Pat. No. 7,249,603; and US 2005/0289582. For purposes of clarification, the terms “physiological measurement,” “biometric expression,” and “biometric data” are used interchangeably herein. Body language, among other things, can non-verbally communicate emotive states via body gestures, postures, body or facial expressions, and the like. Generally, algorithms for physiological measurements can be used to implement embodiments of the present invention. Some embodiments may capture only one or a couple of physiological measurement(s) to reduce costs while other embodiments may capture multiple physiological measurements for more precision. Many techniques have been described in translating physiological measurements or biometric data into an emotional metric data (e.g., type of emotion or emotional levels). See e.g., US 2005/0289582, ¶¶37-44 and the references cited therein. Examples may include Hidden Markov Models, neural networks, and fuzzy logic techniques. See e.g., Comm. ACM, vol. 37, no. 3, pp. 77-84, March 1994. For purposes of clarification, the definition of the term “emotional metric data” subsumes the terms “emotion”, “type of emotion,” and “emotional level.”

Without wishing to be bound by theory, it is generally thought that each emotion can cause a detectable physical response in the body. There are different systems and categorizations of “emotions.” For purposes of this innovation, any set—or even a newly derived set of emotion definitions and hierarchies, can be used which is recognized as capturing at least a human emotion element. For example, refer to Robert Plutchik's defined eight primary emotions of: anger, fear, sadness, joy, disgust, surprise, curiosity, acceptance; or, Paul Ekman's list of basic emotions are: anger, fear, sadness, happiness, disgust. Further well-known is a list by Paul Ekman is his research on facial expressions in humans. Other emotion research focuses on physical displays of emotion including body language of animals and facial expressions in humans.

Other ways to understand emotion includes Dr Albert Mehrabian's P-A-D (Pleasure, Arousal, Dominance) values classification system to model the human emotion state. One explanation is with US2003/0028383A1 where Dr. Mehrabian is a named co-inventor.

The term “body language”, as used herein, broadly includes forms of communication using body movements or gestures, instead of, or in addition to, sounds, verbal language, or other forms of communication. Body language is part of the category of paralanguage, which for purposes of the present invention describes all forms of human or mammalian communication that are not verbal language. This includes, but is not limited to, the most subtle movements of many consumers, including winking and slight movement of the eyebrows. Examples of body language data include facial electromyography or vision-based facial expression data. See e.g., US 2005/0289582; U.S. Pat. No. 5,436,638; U.S. Pat. No. 7,227,976.

The term “paralanguage” or “paralinguistic element(s)” refers to the non-verbal elements of communication used to modify meaning and convey emotion. Paralanguage may be expressed consciously or unconsciously, and it includes voice pitch, volume, intonation of speech, among others. Paralanguage can also comprise vocally-produced sounds. In text-only communication such as email, chat rooms, and instant messaging, paralinguistic elements can be displayed by emoticons, font and color choices, capitalization, the use of non-alphabetic or abstract characters, among others. One example of evaluating paralanguage is provided with the layered voice analysis apparatus, which may include the determination of an emotional state of an individual. One example is described in U.S. Pat. No. 6,638,217. Another example is described in published PCT Application WO 97/01984 (PCT/IL96/00027).

“Layered voice analysis” or “LVA” is broadly defined as any means of detecting the mental state and/or emotional makeup of voice by a speaker at a given moment/voice segment by detecting the emotional content of the speaker's speech. Non-limiting examples of commercially available LVA products include those from Nemesysco Ltd., Zuran, Israel, such as LVA 6.50, TiPi 6.40, GK1 and SCA1. See e.g., U.S. Pat. No. 6,638,217. Without wishing to be bound by theory, LVA identifies various types of stress levels, cognitive processes, and/or emotional reactions that are reflected in the properties of voice. In one embodiment, LVA divides a voice segment into: (i) emotional parameters; or (ii) categories of emotions. In another embodiment, the LVA analyzes an arousal level or an attention level in a voice segment. In another embodiment, voice is recorded by a voice recorder, wherein the voice recording is then analyzed by LVA. Examples of recording devices include: a computer via a microphone, telephone, television, radio, voice recorder (digital or analogue), computer-to-computer, video, CD, DVD, or the like. The less compressed the voice sample, the more likely accurate the LVA will be. The voice being recorded/analyzed may be the same or different language than the investigator's native language. Alternatively the voice is not recorded but analyzed as the consumer/shopper/panelist is speaking.

A potential advantage of LVA is that the analysis may be done without looking at the language of the speech. For example, one approach of LVA is using data with regard to any sound (or lack thereof) that the consumer/shopper/panelist produces during testing. These sounds may include intonations, pauses, a gasp, an “err” or “hmm” or a sharp inhale/exhale of breath. Of course words may also form part of the analysis. Frequency of sound (or lack thereof) may used as part of the analysis.

One aspect of the invention provides using LVA in consumer or market research including consumer analysis. LVA may be used with or without other emotive response indicators or physiological measurements. In another embodiment, qualitative data is also obtained from the consumer/shopper/panelist. Non-limiting examples of qualitative data are a written questionnaire or an oral interview (person-to-person or over the phone/Internet). In one embodiment, at least one facet of the consumer or market research is conducted with the consumer/shopper/panelist at home on the Internet. In yet another embodiment, the consumer/shopper/panelists submits her voice to the researcher via the phone or the Internet. The qualitative data may be subsequently used to support LVA drawn conclusions (such LVA conclusion formed independent of the qualitative data).

In one embodiment, the “passion” a consumer feels for an image, or an aspect of an image, may obtained by the use of a “Passion Meter,” as provided by Unitec, Geneva, Switzerland and described in U.S. patent publication claiming the benefit of U.S. Prov. Appl. No. 60/823,531, filed Aug. 25, 2006 (and the non-provisional US publication claiming benefit thereof). Other examples may include those described in “The Evaluative Movement Assessment (EMA)”—Brendl, Markman, and Messner (2005), Journal of Experimental Social Psychology, Volume 41 (4), pp. 346-368.

Generally, autonomic responses and measurements include but are not limited to changes or indications in: body temperature, e.g., measured by conductive or infrared thermometry, facial blood flow, skin impedance, EEG, EKG, blood pressure, blood transit time, heart rate, peripheral blood flow, perspiration or sweat, SDNN heart rate variability, galvanic skin response, pupil dilation, respiratory pace and volume per breath or an average taken, digestive tract peristalsis, large intestinal motility, and piloerection, i.e., goose bumps or body hair erectile state, saccades, temperature biofeedback, among others. See e.g., US 2007/010066.

In one embodiment, the biometric data comprises cardiac data. Cardio vascular monitoring and other cardiac data obtaining techniques are described in US 2003/0149344. A commercial monitor may include the TANITA, 6102 cardio pulse meter. Electro-cardiography, (using a Holter monitor) is another approach. Yet another approach is to employ UWB radar.

In another embodiment, the biometric data is ocular biometric data or non-ocular biometric data. Ocular biometric data is data obtained from the consumer's eye during research. Examples include pupil dilation, blink and eye tracking data.

Additional physiological measurements can be taken such as: electromyography of the facial, or other muscles; saliva viscosity and volume measures; measurement of salivary amylase activity; body biological function, e.g., metabolism via blood analysis, urine or saliva sample in order to evaluate changes in nervous system-directed responses, e.g., chemical markers can be measured for physiological data relating to levels of neuro-endocrine or endocrine-released hormones; brain function activity. Brain function activity (e.g., location and intensity) may be measured by fMRI, a form of medical imaging in this case directed toward the brain. A non-exhaustive list of medical imaging technologies that may be useful for brain function activity understanding, (but can be used for observing other physiological metrics such as the use of ultrasound for heart or lung movement), include fMRI (functional magnetic resonance imaging), MRI magnetic resonance imaging), radiography, fluoroscopy, CT (computated tomography), ultrasonography, nuclear medicine, PET (Positron emission tomography), OT (optical topography), NIRS (near infrared spectroscopy) such as in oximetry, and fNIR (functional near-infrared imaging).

Another example of monitoring brain function activity data may include the “brain-machine interface” developed by Hitachi, Inc., measuring brain blood flow. Yet another example includes “NIRS” or near infrared spectroscopy. Yet still another example is electroencephalogramy (EEG). See also e.g., U.S. Pat. No. 6,572,562.

It should be appreciated that body language changes and measurements include all facial expressions, e.g., monitoring mouth, eye, neck, and jaw muscles, voluntary and involuntary muscle contractions, tissue, cartilage, bone structure, body limb positioning and gestural activity, limb motion patterns, e.g., tapping, patterned head movements, e.g., rotating or nodding, head positioning relative to the body and relative to the applied stimulus, vocal chord tension and resulting tonality, vocal volume (decibels), and speed of speech. When monitoring body language such as facial expressions or vocal changes, a non-invasive apparatus and method can be used. For example, a video digital photography apparatus can be used that correlates any facial expression changes with facial elements analysis software, or the Facial Action Coding System by Ekman at: http://face-and-emotion.com/dataface/facs/description.jsp or www.paulekman.com. See e.g., US 2003/0032890.

The term “selection preference” refers to a decision made by a consumer for the selection of product as a preference or non-preference, degree of appeal, probability of purchase or use, among others. This can also be additionally thought of as having or choosing an opinion, conscious or unconscious attitudes, whether openly expressed to another individual (via written or oral communication), or not.

The term “query” or “selection preference query” refers to any interaction with a subject that results in them identifying a single stimulus or specific group of stimuli from a broader selection of stimuli. The identified stimulus may be a virtual or physical representation of that stimulus, e.g., package in a real or virtual retail environment, element or that stimulus, e.g., color of packing, scent of product contained in the packaging, picture or text, or a result of using that stimulus, e.g., hair color resulting from hair colorant usage. The “query” or “selection preference query” may be made in any medium, e.g., verbal, oral or written, and may be made consciously, e.g., when probed, or unconsciously, e.g., when a subject behaves automatically in response to given stimulus in a given context. A “query” can result in the selection or deselection of a stimulus; whereas, “selection preference query” results in identification of a stimulus or group of stimuli with positive associations. A “selection preference query” may or may not be related to an intention to purchase.

The term “limited communicative consumer” refers to mammals who cannot articulate meaningfully to researchers. Examples may include a baby who lacks communication development, adult humans with impaired communication abilities (e.g., low IQ, physical handicap), or companion animals (e.g., dogs, cats, horse). Within the human species, the term “limited communicative consumer” refers to babies, some young children, and impaired adults such as from disease, injury or old age condition that possess limited conscious communication skills compared to those of normal human adults. For these consumers, consumer research has found difficulty to ascertain their emotive response and selection preference to products and proposed products.

The term “kinesics” is broadly defined to include the interpretation of body language such as facial expressions and gestures, or non-verbal behavior related to movement, either of any part of the body or the body as a whole. The movement of the body, or separate parts, conveys many specific meanings and the interpretations may be culture-bound and correlate to an emotive state. Many movements are carried out at a sub-conscious or at least a low-awareness level.

The present invention relates to emotive response and selection preference methods to conduct consumer research. It should be appreciated that the present invention can be employed with a test subject when she is evaluating a consumer product, either in a virtual environment or a real environment, wherein the environment (virtual or real) is chosen from a home, office, test facility, restaurant, entertainment venue, outdoors, indoors, or retail store. See e.g., U.S. Pat. No. 7,006,982 B2 (“Purchase Selection Behavior Analysis System and Method Utilizing a Visibility Measure”); US 2002/0161651 A1 (“System and Methods for Tracking Consumers in a Store Environment”); US 2006/0010030 A1 (“System and Method for Modeling Shopping Behavior”); U.S. Pat. No. 6,810,300; U.S. Pat. No. 7,099,734; US 2003/0200129; US 2006/0149634. As a result, the location and use of the emotive response and selection system is not limited to any given environment. The environment can be mobile, such that it can be moved and set up for use in the consumer's home, a retail store, a mall, a mall parking lot, a community building, a convention, a show, and the like. It should also be appreciated that that the emotive response and selection preference systems can comprise a virtual or physical imaging apparatus, or combination thereof, which provides at least one visual stimulus. In one embodiment, the visual stimulus comprises a real store environment. In turn, a “real store environment” means that the environment is non-virtual or real. The store may be one open for business or may be prototypical (for testing). The store may be a mass merchant, drug channel, warehouse store, or a high frequency store to provide a few examples of different store formats.

For example, outside of an in-store retail environment, an imaging apparatus can display visual images, e.g., virtual, photographic, or physical images, of prospective or current product shelf arrangements to conduct consumer research regarding consumer products sold in a retail environment. Such visual imaging may include human representations or avatars such as other product users, shoppers, or employees such as retail store clerks, or other mammals. One advantage of such an imaging apparatus is faster screening and/or deeper insight regarding a consumer's reaction to a particular consumer product since the virtual environment can be realistic to a consumer. A consumer's real-time reaction, upon viewing the consumer product, is one element in determining whether to buy the company's product or a competitor's product is referred to as the First Moment of Truth (FMOT).

Two additional components may also influence the consumer's decision of whether to purchase or not. One is any prior use experience with the product and is referred to as the Second Moment of Truth (SMOT). The SMOT is the assessment of product usage by the consumer or a usage experience by someone else that has been related to the consumer such as by word-of-mouth, internet chat room, product reviews, and the like. In one embodiment, the visual stimulus is static or non-static. In another embodiment, the stimulus comprises the consumer participating (e.g., conducting, observing, etc.) in a task associated with a product's usage. Examples of tasks associated a product's usage may include those described in U.S. Pat. No. 7,249,603 (defining “task”); and 2007/0100666 (listing “activity types” in Table 2B). The SMOT refers to both at the time of product use, and product benefits lasting for a period after product use or application, such as in a use experience, or in product beneficiary situations. Another component is the “Zero” Moment of Truth (ZMOT) which refers to the interaction with a representation of or information about a product outside of the retail purchase environment. ZMOT can take place when the consumer receives or views advertisements, tests a sample (which also then lends some SMOT experience). For a retailer, ZMOT can be pre-market launch trade materials shared by the manufacturer before a product is launched for commercial sale.

FMOT, SMOT or ZMOT can involve aesthetics, brand equity, textual and/or sensorial communications, and consumer benefit, among others. Other factors include the appearance of the product at the point of sale or in an advertisement, the visual appearance (logo, copyrights, trademarks, or slogans, among others), olfactory (smell), and aural (sound) features communicated by and in support of the brand equity, and the graphic, verbal, pictorial or textual communication to the consumer such as value, unit price, performance, prestige, convenience. The communication also focuses on how it is transmitted to the consumer, e.g., through a design, logo, text, pictures, imagery, and the like. The virtual or physical imaging apparatus allows a company to evaluate these factors.

The virtual imaging apparatus gives a company, manufacturer, advertiser, or retailer, the ability to quickly screen a higher number of factors that can affect a consumer's reaction to a product at each or all of the Moments of Truth, e.g., FMOT, SMOT, and ZMOT, and allows for a higher number of consumers to be used in the evaluation of the product. For instance, project development teams within a company can evaluate a large number of consumers and have the data saved in a large database for later evaluation. Another benefit is that the virtual imaging apparatus allows a company to have lower developmental costs since they do not have to continually make costly physical prototypes, i.e., products, packaging, in-store environments, merchandise displays, etc. with virtual renditions. For example, a high-resolution, large-scale imaging apparatus allows a company to generate a virtual computer image, photographic image, or photo-shopped image of various prototypes without physically having to make them.

An additional benefit of the virtual imaging apparatus, when used in conjunction with eye-tracking and an emotive response and selection system, is the ability to detect a consumer's emotive state to a proposed product, advertising slogan, etc. The virtual imaging apparatus allows for improved and faster innovation techniques for a company to evaluate the appeal of various advertising and in-store merchandising elements and/or methods that they employ. The virtual imaging apparatus can be used in a retail store, or, in an in vitro virtual retail environment. See e.g., U.S. Pat. No. 6,026,377; U.S. Pat. No. 6,304,855; U.S. Pat. No. 5,848,399.

Some non-limiting examples of FMOT information that are obtained with the imaging apparatus and emotive response selection system are: actual packaging design such as related to artwork and shape, size, orientation, overall visual package impact, or on-pack promotion; execution in-store such as POP displays, shelf arrangement patterns, shelf-talkers, sampling and demo's; trade sell-in materials such as demo's, information presentation, and business methods; upstream technology products such as direct-to-consumer mobile marketing, electroluminescent product highlighting and display.

In another embodiment, the imaging apparatus is provided with real-life samples for the consumer to pull off a shelf within the same room.

In another embodiment, the image is one that responds interactively with the consumer. See e.g., U.S. Pat. No. 6,128,004.

In another embodiment, the visual stimulus can be supplemented or complimented with an aural sound, such as a jingle associated with a visual advertising slogan, or the slogan itself verbally enunciated. The aural sound can be continuously on or activated only when a consumer picks up or inspects a product. The aural sound can be a sound related to the consumer product, e.g., for laundry detergent, the sound of water may be used. Endless variations could be incorporated dependent upon the desired effect on the consumer.

In another embodiment, the visual stimulus can be supplemented by an olfactory stimulus such as a fragrance, odor, smell, aroma, or flavor. It should be noted that, for the purposes of this description of the present invention, the term “fragrance” is used in the broadest sense and represents a type of “stimulus” that could either be relaxing or stimulating, or perhaps could have a neutral effect on a person. Moreover, the terms “fragrance” or “stimulus” can be interchanged in most cases, with respect to the principles of the present invention. Furthermore, the term “fragrance” can literally represent an actual fragrance (e.g., in a liquid state) or an odor (e.g., in a gaseous state), or the term “fragrance” can represent a flavor (such as in a beverage). The term “fragrance” can also represent essential oils, an aroma or scent.

A “fragrance” can be subliminal (at a concentration too low to be consciously detected by a human) or non-subliminal (at a concentration high enough to be consciously detected by a human). Finally, the terms “fragrance” or “stimulus” can alternatively represent some type of “product.” In the case of a “product”, the terms fragrance/stimulus could represent a perfume or a cologne, for example, or some other complex formulation, e.g., a mixture of two or more perfumes. See e.g., U.S. Pat. No. 4,670,264.

In another embodiment, a tactile or other physical effect stimulus can supplement or compliment the visual stimulus. Examples include samples or representations of tactile or thermal sensation. An example where a thermal sensation stimulus may be helpful in consumer research is when the product is some type of therapeutic device or medical device, for example, such as hot towels, or chemically-activated heat-releasing wraps such as those under the registered trademark THERMACARE®, owned by The Procter & Gamble Company.

The imaging apparatus of an in-store environment allows the consumer to have a natural orientation dedicated to a real-life shopping experience. It also can allow a consumer to give feedback and respond to the imaging apparatus or in-store imaging apparatus in real-time, including with real-scale displayed imagery. For instance, the virtual in-store imaging apparatus can store how many times a consumer picks up a product and places it back on the shelf, how long the consumer looks at the product, and, the precise locations of where the products are chosen by the consumer on the shelf. The virtual in-store imaging apparatus can also be configured to store and monitor all the consumer's responses to the product, e.g., oral, written, physical, or involuntary actions, in addition to data collected by an eye-tracking apparatus. As indicated above, an imaging apparatus can be used with other apparatuses such as an eye-tracking apparatus, head-tracking apparatus, and/or a physiological apparatus that measures at least one physiological response.

The imaging apparatus provides the company, manufacturer, advertiser, or retailer, superior feedback with regard to consumer's behavior and reactions to their products. The vast majority of a consumer's decision-making and emotional reactions to consumer products occurs at the sub-conscious level, and cannot be easily determined by conscious awareness or direct interrogation. By studying, in real-time, variations in the eye-tracking activity and physiological indicator(s) of a consumer (such as electrical brain activity), it is possible to gain insight into what the consumer is sub-consciously thinking or feeling. The level and span of attention, and extent and type of emotions evoked by the product can easily be measured using the disclosed virtual imaging apparatus with the eye-tracking and physiological apparatus. As a result, not only are conscious reactions measured and evaluated but also sub-conscious ones. While real-time study gives the fastest learning, such learning can be done later by returning to stored data of the eye-tracking activity and physiological indicator(s) of a consumer.

Methods of obtaining eye gazing data are described in US 2005/0243054 A1; U.S. Pat. No. 7,046,924; U.S. Pat. No. 4,950,069; U.S. Pat. No. 4,836,670; U.S. Pat. No. 4,595,990. IBM developed a “Blue Eyes” camera capable of obtaining eye gazing data. Eyetracking, Inc., San Diego, Calif. is an example. Video-oculography (VOG) uses see-through goggles to measure eye-in-head position. Techniques may include electro-oculography, corneal reflection, lumbus, pupil, and eyelid tracking, and contact lens. See e.g., US 2005/0243054, col. 4, ¶ 58 et seq. Types of eye gazing data may include eye gaze fixation, eye gaze direction, path of eye gaze direction, eye gaze dwell time. The eye gazing data is relative to the image displayed to the consumer as the data is obtained. The image may be stored or archived during testing by methods well known to archive still and non-still images.

The physiological and imaging apparatus can combine neurological responses, motivational research, and physiological reactions, among others, to provide detailed depth analysis of a consumer's reaction to a product or environment. The levels of arousal, involvement, engagement, attraction, degrees of memorization and brand attribution and association, and indices of predisposition and consideration can all be measured and evaluated with varying levels of degree. The physiological and imaging apparatus allows the company to obtain the degree of arousal and degree of engagement with specificity. In terms of the example shopper analysis model, it is now possible to more accurately and quickly capture an emotive response to a consumer product which may be an element involving opinion formation; and, a probable choice decision element on whether to use, not use, recommend, not recommend, select or not select for purchase. In turn, this allows a company to develop FMOT strategies to stop, hold, and close as it relates to selling a company's product in a store.

The imaging apparatus may additionally or optionally comprise sub-systems. Sub-systems as used herein are units that may be connected to and/or integrated with the imaging apparatus. In addition, or in the alternative, the sub-systems may be connected to and/or integrated with each other in any operative configuration. Sub-systems may contribute to the performance of the imaging apparatus. Non-limiting examples of the sub-systems are described below and include, but are not limited to: physical structures imitating an in-store retail environment or in-home environment, power systems; power inversion systems; control systems; memory systems; sensor systems and safety systems. The imaging apparatus is a powerful tool that can be used in conjunction with the emotive response and selection system.

For example, in one embodiment, the emotive response and selection system comprises at least one imaging apparatus, at least one eye-tracking apparatus used to monitor and track a consumer's eye movements in response to a product, and at least one physiological apparatus that measures a consumer's emotive state or feeling to a consumer product. Collectively, the at least one eye-tracking apparatus and the at least one physiological apparatus form an emotive response apparatus. The at least one image apparatus provides at least one visual stimulus to a consumer. The visual stimulus can be virtual, real, photographic, or holographic, a combination thereof, among others.

It should be appreciated that the visual stimulus can be provided by another apparatus such as a projector, television screen, computer monitor, physical product, or virtual product, among others. It should be further appreciated that the visual stimulus can be a physical form such as a real marketed or prototypical product, package, printed page, website which is optionally displayed on a computer or even accessed via normal internet means, service environment, and the like. The emotive response selection system is used to evaluate a consumer's emotive state at ZMOT, FMOT or SMOT, or a combination thereof. In essence, the emotive response selection system evaluates any behavioral change as expressed through one or more physiological indicators in a consumer resulting from them interacting with a product, whether physical or some virtual representation of the product.

In one aspect of the invention, a single or plurality of additional stimuli can be introduced such as a sense supplement or compliment to the visual stimulus. For example, the supplementary sense stimulus can be introduced at a pre-determined time. One possibility is the use of music or changing the volume or genre of music during the viewing period to determine the impact on eye attention. Another possibility is to have an ad containing a minor element showing swords crossed. During the viewing, background noise or music is playing, and then the sounds of a sword fight may be introduced, with the possibility to determine if more attention is drawn to the minor swords element in the ad. In yet another possibility, halfway through the viewing test period, the essence of flower scents can be introduced to determine how that affects the consumer's viewing attention, especially if there is a flower or flower-related element or reference (graphic or text) used as the visual stimulus.

As a feature of the disclosed emotive response selection system, the measures obtained from the consumer of one or both of the eye-tracking or physiological apparatuses, or derivative analysis of one or both data such as a probable emotive response assignment, can be used, in real-time, to manipulate and change the displayed images. This can be accomplished using software integrated-analysis, or directed by a test observer monitoring the real-time consumer data, among other methods. For example, if it appears that the consumer's attention is drawn to blue products, then, a company or researcher can immediately change their displayed product from red to blue, to evaluate the consumer's reaction. The ability to manipulate, modify, and change the displayed images is a powerful market feedback tool, notwithstanding that the present invention allows a company to do it in real-time. This can be done for not only product color, but shape, text, size, pricing, shelf location or any other possible visual or information form or arrangement. Alternatively, the feedback could be used to change the environment in addition to or separate from the visual stimulus.

One aspect of the invention is to better understand the emotive response element in combination with the attention element of the consumer analysis model in a more covert manner, whether in response to solely visual stimuli or a combination of a visual stimulus with at least one supplemental stimulus. For measuring the attention element, an eye-tracking apparatus or head-tracking apparatus may be used. For measuring the emotive response element, an emotive response apparatus can be used to provide the ability to understand the one or more emotive factors which causes a physiological response and/or change within a consumer. The emotive response apparatus measures at least one physiological measure. A physiological measure may include biological, body language expressed responses, and/or paralanguage, among others.

The probable emotive response is estimated by comparing the physiological measure and optionally the eye-gaze position data with a pre-determined dataset or model that gives probable emotive state or states associated with measures. The use of multiple physiological measures can in some cases be helpful to ascertain probable emotive state or states. Optionally, an output of statistical confidence can be given to each emotive state or aggregate. Optionally, for likelihood weighting if multiple emotive states are probable, a report of likely weighting can be outputted.

Another embodiment of the present invention is to use at least one eye-tracking apparatus and/or head-tracking apparatus with a visual stimulus. For example, a consumer can be shown a computer screen with a portion of a computer-generated store shelving image comprising computer-generated packages sitting on at least one shelf. The consumer sits in front of the computer screen, where a single eye or both eyes' movement are tracked remotely; and, given the known position of the sensors to the computer screen and the known position of the image elements displayed on the computer screen, correlation is possible to know at which element of the visual stimulus that the consumer is casting eye or head direction attention at every measured time.

The eye-tracking or head-tracking apparatus can be worn by the consumer, or, it can be a set of fixed sensors (or known position sensors which are either fixed or moving) remotely located from the consumer that monitors the consumer's eyes and/or head movements when viewing the visual stimulus. The eye-tracking apparatus can further comprise a separate memory device that stores the data obtained from tracking the consumer's eyes and/or head movements, which may be located on the consumer or be remote from the consumer. The memory device can then be electronically or wirelessly connected with a separate computer or storage system to transfer the data. The memory device can further comprise a memory disk, cartridge, or other structure to facilitate the ease of transferring data, e.g., flash memory card. The eye-tracking apparatus can also be configured to wirelessly transfer data to a separate data-capturing system that stores the data, e.g., through Bluetooth technology.

One example of an eye-tracking apparatus that may be used with this invention is the Mobile Eye from ASL which is a tetherless or non-tethered eye-tracking system for use when total freedom of movement is required and video with an overlayed cursor. This system is designed to be easily worn by an active subject. The eye-tracking optics is extremely lightweight and unobtrusive and the recording device is small enough to be worn on a belt. The eye image and scene image are interleaved and saved to the recording device.

In one aspect of the invention, one, two, three, four, five, or more types of the biometric data are obtain from the consumer in a non-tethered manner. “Non-tethered” means the biometric obtaining devices obtain data from the consumer without the consumer having wires or cords or the like attached from the consumer to a stand-alone piece of equipment. The consumer may walk or move around without the restriction (albeit in some embodiments in a confined area such as seated in front of a video monitor) of a tethered wire. For purposes of clarification, wires that are attached to a transmitter that is worn on the consumer's person (such as “wireless microphone”) is still considered “non-tethered” as the term is herein defined. In one embodiment, eye tracking data is obtained by way of a non-tethered means. Other examples of a non-tethered means of obtaining biometric data include a sensing system worn on the consumer's person, such as a wave reflective or transponding sensor, or piece of material that is queried or probed by a remote piece of equipment via for example transmission of an electromagnetic wave that may or may not carry encoded data within the transmitted wave or sequence of waves). In yet another example, the non-tethered means includes the subset means of remotely obtaining biometric data.

In another aspect of the invention, one, two, three, four, five, or more types of biometric data are obtained remotely. The term “remotely” or “remote” means that no biometric data obtaining equipment is on or carried by the consumer to obtain the biometric data. For example, heart data may be obtained remotely by way of UWB radar to sense heart beat or breathing rate. Chia, Microwave Conference, Vol. 3, October 2005. As a further example, UWB has been demonstrated as “see-through-the-wall” precision radar imaging technology, which in this case would remotely sense through a human vision barrier. In one embodiment, eye gazing data is obtained in a remote manner. One example may include the use of remote cameras to eye track the consumer to obtain eye gazing data.

Without wishing to be bound by theory, the use of non-tethered obtaining data provides better data from testing given that testing environment is more analogous to “real life” since consumers typically do not have distractive or cumbersome equipment on their person or tethered to equipment. It also facilitates other avenues of testing which may require the consumer to participate in product usage or visit a retail store (commercial or prototypical) that do not lend themselves well to tethered methods.

To measure the emotive state of the consumer, at least one physiological apparatus is used. For example, the physiological response of a consumer's blood pulse can be taken when viewing the visual stimulus while eye-tracking data is simultaneously gathered. The measured data from the physiological apparatus is synchronized in time with the element to which the viewer has directed her attention at a point in time or over a period of time by computer software. While the recording of clock time is valuable, synchronization does not necessarily need to tag with actual clock time, but associate data with each other that occurred at the same point or interval of time. This allows for later analysis and understanding of the emotive state to various elements along the consumer's eye-gaze path. Another aspect of this invention is that certain emotive measurements, e.g., blood pulse measures, can be used to indicate topics or areas, e.g., visual elements, for later research such as a questionnaire if the measurement value(s) meets, exceeds or is less than some pre-determined level set by the researcher.

The physiological apparatus can be worn by the consumer, or, it can be a set of fixed sensors or single sensor remotely located from the consumer that monitors the physiological responses of the consumer when viewing the visual stimulus. For example, the physiological apparatus can be a remotely located infrared camera to monitor changes in body or facial temperature, or the apparatus may be as simple as a watch worn on the wrist of the consumer to monitor heart rate. It should be appreciated that in an exemplary embodiment, the physiological apparatus is a wireless physiological apparatus. In other words, the consumer is not constricted by any physical wires, e.g., electrical cords, limiting their movement or interaction with the visual stimulus.

The physiological apparatus can further comprise a separate memory device that stores the data obtained from tracking the consumer's physiological changes, which may be located on the consumer or be remote from the consumer. The memory device can then be electronically or wirelessly connected with a separate computer or storage system to transfer the data. The memory device can further comprise a memory disk, cartridge, or other structure to facilitate the ease of transferring data, e.g., flash memory card. The physiological apparatus can also be configured to wirelessly transfer data to a separate data-capturing system that stores the data, e.g., through Bluetooth technology. Either way, the end result is that the data from the eye-tracking apparatus and the physiological apparatus is transferred to a separate apparatus that is configured to correlate, evaluate, and/or synchronize both sets of data, among other functions. For purposes of a simplified description, the separate apparatus is described as a data-capturing apparatus. The data-capturing apparatus can be a separate computer, a laptop, a database, server, or any other electronic device configured to correlate, evaluate, and/or synchronize data from the physiological apparatus and the eye-tracking apparatus.

The data-capturing apparatus can further comprise additional databases or stored information. For example, known probable emotive states associated with certain physiological or eye-gaze measurement values, or derivative values such as from intermediate analysis, can be stored and looked up in a table within the database and then time-associated, i.e., synchronized, with the viewed element for each or any time interval, or over a period of time, recorded during the period that the consumer is viewing the visual stimulus. It should be appreciated that a given physiological measure can also indicate two or more possible feelings either singly or in combination. In these cases, all possible feelings can be associated with a given time interval in the database.

Another additional database or stored information can be known selection states associated with certain emotive states, physiological, or eye-gaze measurement values, or derivative values such as from intermediate analysis, which can be stored and looked up in a table within the database and then time-associated, i.e., synchronized, with the viewed element for each or any time interval, or over a period of time, recorded during the period that the consumer is viewing the visual stimulus.

In another aspect of the invention, the measurement and tracking with subsequent time-association entry into the data-capturing apparatus of multiple physiological data such as a blood pulse measurement and a voice measurement is possible. For the measured values, a feeling or possible feelings or emotive state(s) can then be assigned for each and associated time interval in the database. The recorded feeling(s) for each can be compared to each other to output a new value of a most likely feeling or emotive state, based on cross-reinforcement of the individual database ascribed feelings, or an analysis sub-routine based on a prior model or correlation created beforehand with the emotive response measures involved. In other words, the data obtained from the eye-tracking apparatus and physiological apparatus, can be used in conjunction with other databases storing information in the data-capturing system to output processed data. The processed data is in a synchronized format.

In all cases, whether one or multiple emotive states are measured, the assigned feelings from models, correlations, monographs, look-up tables and databases and the like, can be adjusted internally for a specific consumer, or different environmental factors known or surmised to modify the feeling/emotive value correspondence can also be used. In some cases, a “control” measure conducted in advance, during or after the viewing test such as a specific consumer's response to controlled stimuli, questions, statements, and the like, can be used to modify the emotive value correspondence in that case. Alternatively, a specific physiological response profile(s) modeled beforehand can be used as the “control.”

The emotive response and selection system can also be used to provide data for the third element of the example shopper analysis model, i.e., the selection preference element (also known as the purchase-intent or product choice element. In other consumer analysis models, this corresponds to product preference or willingness to recommend for use for one's self or someone else). Similar to the example provided above, the consumer examines various products and product elements and a separate database captures the time-associated data of eye-tracked attention focus on which element, the physiological measure(s), and the likely emotive state associated with the physiological value or combined eye-tracked analysis and physiological measures are outputted.

This aspect of the invention comprises a step or plurality of steps to help determine the probability of the consumer's selection preference for the consumer product, based on a database or stored information of known probable selection states associated with certain emotive states, physiological, or eye-gaze measurement values, or derivative values such as from intermediate analysis, or a combination thereof. An additional selection preference step can be accomplished by asking the consumer during the viewing period of her degree of choice decision about any consumer product, or in this case, inquire about such as purchase intent. Such questioning (by written or verbal methods) can be done concurrently with the visual stimulus applied, or after the viewing exercise, e.g., either right afterwards or at a postponed later time frame. While not required, during such questioning, the physiological apparatus can continue to collect data to help gauge the veracity of the consumer's response to such inquiries.

The latter inquiry step is optional with ZMOT, FMOT or SMOT research. If a database or stored information of known probable selection states associated with certain emotive states, physiological, or eye-gaze measurement values, or derivative values such as from intermediate analysis, or a combination thereof, is not available, then the inquiry step can be used to collect consumer-expressed selection preference states and associated with collected emotive states, physiological, or eye-gaze measurement values, or derivative values such as from intermediate analysis. This provides a method for creating a model or database or table of at least one input of emotive states, physiological, or eye-gaze measurement values, or derivative values with at least one output of at least one probable or likely selection preference. It also can provide a degree of conviction for a selection preference. For example, for purchase intent one can ask for degrees of preference such as: very likely to purchase, likely to purchase, unsure or uncertain, likely not to purchase, or very likely not to purchase, among others. Once the model is available, subsequent consumer research with consumers can provide a likely selection preference without the need for a query step during the consumer research.

Also, at the option of the researcher, she can import from someone else or create her own data or look-up table for use in the emotive response and selection preference system for the particular test, and emotive response analysis. As an example, a researcher could expose a panelist to set of different stimuli, measure one or more physiological indicators and then in a sequential or concurrent manner consciously probe or query the panelist to determine the consciously expressed emotive state(s). A non-limiting example can be found in U.S. Patent Pub. No. 2003/0236451A1.

Alternatively, instead of employing a probe or query to help build the model, a second physiological measure can be concurrently measured and synchronized with the first, whereas with the second measure, the data, model or look-up table for its transformation into likely emotive state(s) is previously known. Because of the synchronous link between the two physiological measures, the known model with the second physiological measure, can be used in determining a emotive state model for the first.

Moreover, different or additional physiological apparatus can be employed in conjunction with the emotive response and selection preference system. They can be employed as one or more of the physiological measures as part of the visual stimulus that is coupled with eye-tracking apparatus data, or employed in a couple ways with the selection preference query. For example, a layered voice analysis apparatus can be used when verbal inquiries of purchase intent are asked of the consumer by recording the consumer's voice at known question response times, and then comparing the data to tables of corresponding feeling(s), by which both the veracity and degree of enthusiasm for the expressed opinion can be estimated. The layered voice analysis may examine and record data from the intonation of the consumer's response to the inquiries. U.S. Pat. No. 6,638,217 discloses a layered voice apparatus that may be used with the present invention. In another embodiment, the emotional metric data, the eye tracking data, the biometric data, other relevant data, can be used in any combination to provide an estimation or probability of purchase intent by the consumer of the visual stimulus.

It should be appreciated, however, that the components comprising the emotive response and selection preference system can be used separately or on conjunction with each other. The emotive response and selection preference system provides at least one benefit of synchronized understanding of a consumer's emotive response to a proposed product, package, advertising copy/slogan, or merchandising proposition, i.e., a shelf display, either post-test or in real-time.

In another embodiment, the emotive response and selection preference system may comprise any combination of the following four elements: (1) at least one visual stimulus element; (2) at least one eye-tracking or head-tracking apparatus; (3) at least one non-pupil or non-ocular physiological measurement apparatus; and (4) at least one apparatus configured to synchronize the data from elements 2 and 3.

Referring now to FIGS. 1A-1E, the first element provides at least one visual stimulus 1 to a consumer. The first element can provide a plurality of visual stimuli to a consumer, if desired. In one embodiment, the stimulus is provided by an imaging apparatus as disclosed above. In another embodiment, the stimulus can be a physical representation of the consumer product. Additional stimulus can be used in conjunction with the visual stimulus such as olfactory, aural, taste, or tactile in nature, among others. The visual stimulus can be a physical manifestation or a virtual representation. The stimulus can be presented in any environment, such as a ‘sterile’ setting to an ‘in-store’ setting, or even in an ‘at-home’ setting. The visual stimulus can also be a physical activity such as shopping, washing clothes, picking up a consumer product, or smelling a consumer fragrance. The visual stimulus can also be provided when one or more of the other consumer's senses are ‘blinded’ or limited in some fashion. For example, a consumer can only see and taste the consumer product rather than being able to smell it.

The second element is an eye-tracking 2 or head-tracking apparatus as described above. The eye-tracking apparatus 2 can measure and monitor both eyes of a consumer, independently or together, or a single eye if one eye is blind-folded or limited in its ability to physically view the visual stimulus. The eye-tracking apparatus 2 can be substituted or used in conjunction with a head-tracking apparatus (not illustrated). In one embodiment, the eye-tracking apparatus 2 and/or head-tracking apparatus is a wireless apparatus physically placed on the consumer (FIG. 1B). In another embodiment, the eye-tracking apparatus 2 is a head-mounted unit, or can be as simple as a pair of glasses worn by the consumer, which includes at least one video camera providing video image of the field of view of the consumer to which the eye-gaze position is referenced.

In still yet another embodiment, the eye-tracking apparatus 2 and/or head-tracking apparatus is a set of fixed sensors spatially separated from the consumer, e.g., mounted on a wall with visual images the consumer is viewing, wherein the displayed imagery is to which the eye-gaze position is referenced. The wireless eye-tracking or head-tracking apparatus can transmit data to a stand-alone electronic apparatus 3, i.e., a computer, laptop, or electronic database, separate from the wireless apparatus (FIG. 1D). Data can also be physically stored in the wireless eye-tracking or head-tracking apparatus, i.e., using a flash memory card, for later download to the data-capturing apparatus 3 (FIG. 1E), or to an intermediate information storage apparatus 4.

Referring back to FIGS. 1A-1E, the third element is at least one non-pupil physiological measurement apparatus 5, i.e., a physiological apparatus. For visual stimulus research, a non-pupil (that is, non-eye or non-ocular) physiological measure can be selected to avoid possible concerns on pupil response based on other visual light factors such as intensity, clarity either associated with the visual stimulus, image apparatus or environmental lighting. The physiological apparatus 5 measures at least one physiological response of the consumer, e.g., an autonomic response. The physiological apparatus 5 can measure a single physiological measure and any associated change or a plurality of physiological measures, and any associated changes. In one embodiment, the physiological measurement apparatus is a wireless apparatus physically placed on the consumer FIG. 1C, e.g., electrodes placed on the skin of a consumer. In still yet another embodiment, the physiological measurement apparatus is a set of fixed sensors spatially separated from the consumer, e.g., an infrared camera mounted on a wall. Similar to a wireless eye-tracking 2 or head-tracking apparatus, the wireless physiological apparatus 5 can transmit data to a data-capturing apparatus 3 separate from the wireless apparatus 5 (FIG. 1D), or, can physically store data in the wireless apparatus 5 for later download to an intermediate information storage apparatus (FIG. 1E). It should be appreciated that there are no wires, e.g., electrical, restricting the movement of the consumer. The physiological measurement apparatus 5 is also very mobile allowing the consumer to easily move around.

Autonomic responses and measurements include body temperature (conductive or IR thermometry), facial blood flow, skin impedance, EEG, qEEG (quantified electroencephalography), EKG, blood pressure, blood transit time, heart rate, peripheral blood flow, sweat, SDNN heart rate variability, galvanic skin response, pupil dilation, respiratory pace and volume per breath or an average taken, stomach motility, and body hair erectile state, among others. Additional physiological measurements can be taken such as a facial electromyography, saliva viscosity and volume, measurement of salivary amylase activity, body metabolism, brain activity location and intensity, i.e., measured by fMRI or EEG

The fourth element is a data-capturing apparatus 3, i.e., a computer, laptop, server, or electronic database, among others, which can correlate, evaluate, and/or extrapolate data obtained from the first, second and third elements of the emotive response and selection preference system. The data-capturing apparatus 3 also synchronizes the data from the first 1, second 2, and third 5 elements. The data-capturing apparatus 3 may comprise a single database or a plurality of databases that stores the data. The data-capturing apparatus 3 can evaluate or estimate the change or nature of the mood and/or attitude of the consumer, among other things pertinent to consumer research, using a software analysis program, if desired. The data-capturing analysis can compare the captured data from the emotive response and selection preference system with stored pre-determined models and data. In another embodiment, an intermediate information storage apparatus 4 is used to transfer data to the data-capturing apparatus 3.

There can be any combination of the following steps in measuring a consumer's emotive state with the disclosed emotive response and selection system. These steps may include: (1) providing at least one visual stimulus to a consumer; (2) measuring and recording the movement of at least one eye of the consumer; (3) measuring at least one physiological element from the consumer; and (4) synchronizing the eye-tracking data with the physiological data to determine the emotive state of the consumer by comparing the synchronized data with a pre-determined model or database of probable emotive states. A company can then use the synchronized data (eye-tracking data and measured at least one physiological data) to evaluate and pinpoint a change or reaction in the consumer's affective or emotive state towards the visual stimulus or an element of the stimulus, e.g., target product, slogan, and the like. In another embodiment, a company or researcher can use the synchronized data as feedback to control and/or manipulate a consumer's affective or emotive reaction or response towards the target product, slogan, and the like.

It should be appreciated that a plurality of visual stimuli could be applied, sequentially, or all at once, and/or non-visual stimulus or stimuli could be applied in conjunction or separately from the applied visual stimulus. Each time a new stimulus is introduced and/or changed, the consumer's physiological response is monitored and captured. The visual stimulus can be viewed by the consumer on a computer monitor, plasma screen, LCD screen, CRT, projection screen, fogscreen, water screen, or any other structure, e.g., imaging apparatus, that allows a real or virtual image to be displayed. The visual stimulus can also be a physical representation.

In yet another embodiment, a consumer questionnaire is presented to the consumer and obtaining an answer thereto, wherein the questionnaire comprising one or more psychometric, psychographic, demographic questions, among others, can be asked. The answers can be obtained before, during, after, or combination thereof at the time of presenting the visual stimulus to the consumer. The emotive response and selection preference system can further obtain feedback from the consumer's response to the questions asked, with the questions optionally asked after the test and then obtained at that or a later time by the emotive response and selection system. The data can also be correlated with psychometric measurements such as personality trait assessments to further enhance the reliability of the emotive response and selection preference system and methods.

In still yet another embodiment, the emotive response and selection preference system provides a company or researcher the ability to evaluate and monitor the body language of a consumer after he/she views a consumer product with the physiological apparatus. The emotive response and selection preference system provides a company the ability to understand and critically evaluate the body language, conscious or unconscious responses, of a consumer to a consumer product. The physiological apparatus can measure a single body language change or a plurality of body language changes of a consumer. Body language changes and measurements include all facial expressions, i.e., monitoring mouth, eye, neck, and jaw muscles, voluntary and involuntary muscle contractions, tissue, cartilage, bone structure, body limb positioning, hands, fingers, shoulder positioning and the like, gestural activity, limb motion patterns, i.e., tapping, patterned head movements, i.e., rotating or nodding, head positioning relative to the body and relative to the applied stimulus, vocal chord tension and resulting tonality, vocal volume (decibels), and speed of speech. When monitoring body language such as facial expressions or vocal changes, a non-invasive physiological apparatus and method can be used. For example, a video digital photography apparatus can be used that captures and may correlate any facial expression change with facial elements analysis software.

In a preferred embodiment of the emotive response and selection preference system, the at least one physiological apparatus is a physiological telemetric apparatus 5 illustrated in FIG. 1C. The physiological measuring apparatus uses at least one wireless, or un-tethered physiological measure sensor to monitor changes in the physiology of the consumer, e.g., biological, autonomic responses, body language, paralanguage, vocal, and the like. The physiological measure sensor can be a heart rate monitor or other electronic device that measures physiological changes of or within a consumer. The physiological telemetric system 5 can measure a change in a consumer's physiology while the consumer performs a specific task or uses a product such as during SMOT research, or when a visual stimulus is applied such as during ZMOT or FMOT research. For example, the consumer can shop in an in-store retail environment, in a virtual retail environment using the imaging apparatus disclosed above, view proposed magazine advertisement layouts for a product, shop in a physical store for pet food, peruse a website providing tips and savings coupons for oral care products, wash clothes at home, clean at home, or other tasks that are related to the consumer product that is being evaluated by the company.

Other embodiments may include those described in US 2005/0289582 A1, ¶¶15-23.

In one aspect of the invention, the consumer is presented with questions soliciting attitude and/or behavioral data about the visual stimulus. See e.g., US 2007/0156515.

In another aspect of the invention, the data of the present invention may be stored and transferred according to known methods. See e.g., US 2006/0036751; US 2007/0100666.

### Example 1

A prototype rendering of a proposed magazine advertisement is shown to a consumer. Using available eye-tracking apparatus systems, as illustrated in FIGS. 1A-1E, the noticeability of various elements within the magazine advertisement layout is measured. The consumer's eyepath is overlaid on a copy of the ad image, where both the sequence as well as time spent viewing each element is recorded and displayed. The sponsor of the ad then shows the consumer various ad layouts or elements different from the first ad layout to gauge their reactions. For example, the sponsor may learn that one key graphic receives little notice while another aspect of the ad receives an inordinate amount of attention, both in terms of total time and number of revisits by the consumer's eyes during the test viewing period. In another version of a test, two pages of a simulated magazine may be concurrently shown where one is an ad for the sponsor's product or service and the other is an ad for a competitor's product or service. The view path sequence, the amount of time devoted by a viewer with one ad versus the other can help the sponsor to understand which ad will draw more attention among magazine readership. This information can also help motivate the sponsor to try to improve the attention-appeal of their ad design by modifying it.

Beyond attention understanding, the emotive response and selection preference system provides even more information to better understand the consumer's emotive reaction to the two simulated pages of magazine advertisement. In addition to the use of an eye-tracking apparatus during the viewing, a physiological apparatus is used to monitor at least one physiological indicator. For example, a facial digital videography apparatus is focused upon the consumer's face to record facial expression. The facial expression data is then assigned a probable state of expression such as a “moderate smile” and then transformed to a probable feeling such as “pleased”, based on a stored lookup table for both derivative outputs with the digital facial expression data. The eye-tracking data, facial expression data and the derivative emotive response outputs are associated in a synchronized way.

The collective history of the viewing session can be collected and later reviewed or outputted in a report for the researcher to better understand the emotive reaction by the consumer. While the range of human emotions and states can be multi-dimensional, processed and reported in a variety of manners as befits the researcher's desires, for this example, the emotive state reported is a degree of pleasure to displeasure. The eye-track path and the time spent at each key stopping point is shown along with the corresponding degree of pleasure or displeasure. Optionally, in this example, the emotive state or the raw facial expression data can be associated with a pre-determined selection preference lookup table. The selection preference is expressed as a degree of like or dislike. Both the emotive state understanding and the selection preference understanding can help increase the sponsor's selection of the best advertisement for their business.

### Example 2

As a modification of Example #1, instead of the stimulus being an image of a magazine ad, a virtual rendering of a retail store shelf with different products is displayed. In a manner similar to Example #1, the attention, physiological and derived emotive state data can be determined. Additionally, the probable selection preference of the consumer, for example purchase intent, toward different products that received her attention can be assigned and reported either in real-time or in a post-test report.

### Example 3

One non-limiting SMOT example is a consumer changing a diaper on a real or doll baby. Eye-tracking or head position apparatus is employed, while a physiological measure of the mom is collected, such as a digitized voice recording of audio narration provided by the mom during the diaper changing task. Synchronizing data plus employing layered voice analysis yields probable emotive state(s) through the task, including points of frustration and pleasurable execution. Giving the mother a different diaper design, the SMOT data and emotive response profiles from the second diaper can be compared with the first. This allows the company to understand differences between the different diaper designs, and thereby design improved diapers and diapering experiences.

In diaper changing, two individuals are often involved, one being a baby. The baby's emotive response profile toward the diaper and the diaper changing experience, can be estimated or determined. For a baby, one physiological measure is to monitor at least one of the baby's physiological signs to understand the stresses, and other factors of diaper changes upon them. For example, a remote sensor infrared camera can track the baby's skin surface temperature changes, i.e., focusing on the facial region. Head-position or eye-tracking apparatus is optionally employed. The physiological data and optionally-derived emotive state(s) is optionally synchronized with event tags which indicate at what part of the diaper changing task the data corresponds or represents. Such tags can be determined by an observer or vision system analysis, among others. For this example, the average or ending physiological data or emotive state of the baby may be a useful reported output by which a diaper manufacturer can: (i) determine the most pleasurable protocol for changing diapers and share that with all consumers & pediatricians; or, (ii) design better diapers.

### Example 4

In food research, the appearance of food can be important, including with processed foods. For example a green colored version of a slice of American cheese may garner less preference by human consumers than a similar slice of American cheese to which the only difference is color, with the second yellow-orange slice exhibiting the traditional appearance. In other mammals, such as dogs, food appearance can be important at times as well. Food appearance can take in a whole range of appearance factors; however, for this example the variable will be the color of dry dog food whereas all other visual cues such as food pellet size, shape, visually discernable texture are kept the same. Further, the food ingredients composition, method of preparation, cooking manufacture are the same, so that other possible cues, such as emitted food odor, are the same or similar.

In this example, a dog consumer has a heart-beat sensor affixed to its body, similar to such sensors affixed to humans, where the measured heartbeat data is wirelessly transmitted to a remote data storage device. Head position apparatus is also employed to collect head position data. The heartbeat can indicate degree of excitement or arousal. In one test, a pair of bowls of dry dog food is placed in front of the dog consumer, where the only difference is that the color appearance of the food is different between the two. As the dog gives attention to one or both bowls, the head position data is tracked, concurrent heart beat data collected and synchronized which is then transposed to the emotive response or emotive state vector of excitement or arousal. Just as important to this case, and others, may be noted the amount of disinterest, such as if the consumer spends little or no time with its head position to either bowl.

The next stage of the test can be query of selection preference whereas the consumer is free to physically approach the bowls and choose one for taste sampling and or eating. The consumer dog may have been prevented from previously approaching the bowls by owner command or a temporarily positioned intervening screen.

### Example 5

In home cleaning, floors are often cleaned via use of a dry mop. To better understand the consumer use experience of cleaning a room floor with a dry mop with affixed nonwoven cleaning sheet, a consumer is affixed with head-mounted eye-tracking equipment as well as a physiological sensor. Both the eye-tracking apparatus and the sensor wirelessly transmit data to a remote data storage device. The consumer is introduced to a physical room in a test facility where the positioning of furniture and the location and amount of dirt on the floor are test variables set by the researcher. The consumer then cleans the room while the data provides the researcher a continuous feed of the focus of eye gaze attention, and a physiological data stream converted to probable emotive state(s), such that the researcher can understand the emotive response while the consumer cleans certain parts of the floor, such as when working the mop in the open floor area versus around certain pieces of furniture.

Additionally, these learnings can also be associated with the measured amount of dirt collected, a product performance measure, on the cleaner sheet by the consumer, as a percentage of the mass of dirt initially distributed on the test floor before by the researcher. As another variation, different panelists can be exposed to no aural stimulus, silence, or sound (e.g., music) during the task to determine its effect on emotive response. Or for the same panelist, music and silence can be alternated to determine effect. As a further variation, instead of introduction of music versus none, scent can be introduced at certain periods versus none (or versus a different scent), again to evaluate effect on the consumer's cleaning experience.

### Example 6

Post-application beneficiary analysis. An example of this is an adult consumer that applies a shampoo product on their hair, and then is the subject of consumer analysis eight (8) hours later to determine their emotive response and possible preference to the presence and degree of one or more product benefits, such as hair shine, hair color, hair feel, hair manageability, and the like. Using the same example, if the same adult consumer applies the shampoo to a child's head, then the beneficiary may be the child, as well as the adult who may be the child's mother. In that case, one or both beneficiaries may be subject of beneficiary research.

While several examples are given above, they are not to be limiting in any fashion. Many situations will find this methodology useful, such as in consumer research on ease of package using (SMOT), POP media selection (FMOT), or billboard appeal (ZMOT), laundry detergent usage in-home (SMOT).

In still yet another embodiment, the emotive response selection and preference methods are used to conduct consumer research on a plurality of consumers at the same time. Previously, consumer research could only be conducted with a single consumer; however, the present invention allows a company to conduct consumer research on a plurality of consumers; thus, increasing the speed at which consumer research is conducted while also increasing the quality and efficiency of doing consumer research. One way to effect this is to use an eye-tracking apparatus and a physiological apparatus for each consumer in a group of consumers.

The emotive response and selection preference methods gives a company, manufacturer, advertiser, or retailer, superior feedback with regard to consumer's behavior and reactions to their products. The exhaustive results, which are obtained from the elements comprising the emotive response and selection preference system, provide an in-depth understanding of a consumer's habits and feelings such as during shopping, viewing, usage or post-usage benefit. Optionally, there is a behavioral (physiological) and query (questionnaires) component with the disclosed emotive response and selection preference system. The query component can be conducted in real-time, before, or after the consumer has been exposed to the in vitro environment, or an actual in vivo environment such as a physical store or actual journal reading or website perusal.

It should also be appreciated that the methods of the present invention may also contemplate the step of applying a visual stimulus to a consumer through an eye-tracking device when it is worn. In this manner, the consumer does not need to be in a retail environment, if desired. In another embodiment, an image flipper, e.g., to allow a mirror video image to be displayed, can be used to better understand the personal hygiene and beauty tasks of a consumer. For example, the consumer's own image is captured by video and displayed back to the consumer in real-time onto a visual screen (e.g., video monitor) after image flipping such that it appears to the consumer that they are viewing themselves in a physical mirror. Eye-tracking apparatus concurrently capture eye-tracking data and optionally biometric data is obtained and is typically not displayed in the image provided to the consumer, but is preserved for viewing by the researcher later. In this embodiment, the researcher can observe in real-time or later where the consumer is looking as they apply skin care, hair care, cosmetics, and other products to their body or face, or perform tasks such as shaving and oral hygiene.

One aspect of the invention provides for defining an area of interest (AOI) in the visual stimulus that is presented to the consumer. The AOI may be defined by the investigator for numerous reasons. Some non-limiting reasons may be to test a certain characteristic of a product, or part of a graphic in an advertising message, or even a stain on a floor while the consumer performs the task of scrubbing the stain with a product. Alternatively, the AOI may be defined, at least in part, by data (e.g., eye gaze duration in an area of the visual stimulus.)

The visual stimulus and AOI's, for reporting purposes of the investigator, may be illustrated as a graphic. The graphic may be an archived image of the visual stimulus or some other representation. In turn the AOI may be illustrated on the graphic by drawing a circle or some other indicium indicating the location or area of the AOI in the graphic (“AOI indicium”). Of course a visual stimulus (and the graphic of the visual stimulus) may comprise a plurality of AOI's (e.g., 2-10, or more). Each AOI (and thus AOI indicium) need not be uniform in size.

Upon defining the AOI, the researcher may collect biometric data and eye gazing data from the consumer while presenting the visual stimulus to the consumer. By temporally sequencing the collected eye gazing data in relation to the AOI, the researcher can determine when the consumer's gaze is directed within an AOI and thus associate the collected eye gazing data and the collected biometric data in relation to the AOI. Of course biometric data can be translated to emotional metric data before or after being associated with collected eye gazing data (in relation to the AOI). One skilled in the art will know to take into account any “lag time” associated with the biometric data and the emotional response and/or eye gaze data. For example, a cardiac data will often have a lag time (versus say brain function activity data which is essentially or nearly instantaneous).

In one embodiment, the investigator may compare biometric data/emotional metric data/eye gazing data in relation to a first AOI to that of the data in relation to second AOI, and a third AOI, and the like. The emotional metric data or biometric data in relation to the AOI may be presented on a graphic (comprising the visual stimulus) as an indicium. The indicium may be simply presented as raw data or perhaps a symbol (e.g., a needle on a scale) or scalar color-coding or scalar indicium size or the like. The indicium may also communicate a degree of statistical confidence or range or the like for either the emotional metric or biometric data. There may be more than one indicium associated with a given AOI, such as two different biometric or emotional metric or combination indicia; or, indicium based on data from different consumers or the same consumer but in two different time-separated tests. The indicium may represent positive or negative values relative to the specific metric chosen by the researcher. Additionally, the indicium can represent the collection of multiple consumers such as an average, a total, a variation from the mean, a range, a probability, a difference versus a standard, expectation or project goal of the data, as a percentage or number of consumers with data or data that falls within a defined set of limits or a minimum or maximum defined value. Optionally, the eye-gaze path or sequence of viewing may also be shown in whole or part. Of course the researcher may choose to present the data obtained (according the methodologies herein) described by presenting the data in a report that comprises: a graphic of the visual stimulus; an area of interest (AOI) indicium; an emotional metric data indicium or a biometric data indicium regarding the AOI; and an eye gazing indicium regarding the AOI. The report may be a hardcopy or presented electronically.

The emotive response and selection preference methods described above merely illustrate and disclose preferred methods of many that could be used and produced. The above description and drawings illustrate embodiments, which achieve the objects, features, and advantages of the present invention. However, it is not intended that the present invention be strictly limited to the above-described and illustrated embodiments. Any modification, though presently unforeseeable, of the present invention that comes within the spirit and scope of the following claims should be considered part of the present invention.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of obtaining consumer research data comprising the steps:
(a) presenting a visual stimulus to a consumer, wherein the visual stimulus comprises a vertically flipped image of the consumer;
(b) collecting eye gazing data from the consumer while presenting the visual stimulus to the consumer.

2. The method according to claim 1 further comprising the step of collecting biometric data from the consumer while presenting the visual stimulus to the consumer.

3. The method of claim 2, wherein the method further comprises the step of translating the collected biometric data to an emotional metric data.

4. The method of claim 2, wherein the biometric data is chosen from brain function activity data, voice recognition data, body language data, cardiac data, ocular data or combinations thereof.

5. The method of claim 2, wherein the biometric data comprises paralanguage.

6. The method of claim 1, wherein the visual stimulus comprises the consumer participating in a task associated with personal grooming and/or a product's usage.

7. A method of obtaining consumer research data comprising the steps;
(a) presenting a visual stimulus to a consumer, wherein the visual stimulus comprises a vertically flipped image of the consumer;
(b) defining an area of interest (AOI) in the visual stimulus;
(c) collecting eye gazing data from the consumer while presenting the visual stimulus to the consumer;
(d) collecting biometric data from the consumer while presenting the visual stimulus to the consumer; and
(e) associating the collected biometric data and the collected eye gazing data in relation to the AOI.

8. The method of claim 7, further comprising the steps:
(a) indicating the AOI as an AOI indicium on the graphic;
(b) indicating the associated collected biometric data as a biometric data indicium on the graphic;
(c) indicating the associated collected eye gazing data as an eye gazing data indicium on the graphic.

9. The method of claim 7, wherein the biometric data is chosen from brain function activity data, voice recognition data, body language data, cardiac data, ocular data, or combinations thereof.

10. The method of claim 7, wherein the biometric data comprises paralanguage.

11. The method of claim 7, further comprising the step of obtaining answer(s) from the consumer in response to a consumer questionnaire comprising one or more psychometric, psychographic questions.

12. The method of claim 7, further comprising the step of collecting face direction data from the consumer while presenting the visual stimulus to the consumer.

13. The method of claim 7, wherein the visual stimulus comprises the consumer participating in a task associated with personal grooming and/or a product's usage.

14. Apparatus for capturing consumer behavior comprising:
a) an image capturing element disposed to capture at least a single image of a consumer;
b) an image flipping element adapted to flip a captured image along a vertical axis creating a mirror image of the consumer;
c) an image display element disposed to display the flipped image to the consumer;
d) an eye-tracking element disposed to capture consumer eye-tracking data as the consumer views the displayed image.

15. The apparatus according to claim 14 further comprising a physiological measurement collection element.

16. The apparatus according to claim 14 wherein the eye-tracking element is un-tethered.
